(19) United States
(12) Patent Application Publication
(10) Pub. No.: US 2018/0289504 A1
(43) Pub. Date: Oct. 11, 2018

(54) FENESTRATED SPINAL IMPLANT

(71) Applicant: Quandary Medical, LLC, Denver, CO (US)

(72) Inventors: Brandon Arthurs, Wilmington, NC (US); Scott Noble, Denver, CO (US); Ryan Arce, Denver, CO (US); Leighton LaPierre, Thornton, CO (US); Jeffrey Schell, Denver, CO (US)

(73) Assignee: Quandary Medical, LLC, Denver, CO (US)

(21) Appl. No.: 15/950,940

(22) Filed: Apr. 11, 2018

Related U.S. Application Data
(60) Provisional application No. 62/483,974, filed on Apr. 11, 2017.

(51) Int. Cl. A61F 2/44; A61F 2/30; A61F 2/28

(52) U.S. Cl. CPC A61F 2/4455; A61F 2/442; A61F 2002/30774; A61F 2/28; A61F 2002/4475; A61F 2/30771

(57) ABSTRACT

The present invention relates to an implant, and more precisely in the preferred embodiment, a fenestrated non-distracting rod for use in association with spinal surgery. The implant is to be placed via the pre-sacral approach in an embodiment of the invention. The fenestrations incorporated within the preferred embodiment are configured to collect bone during transit through the sacrum, and then optionally configured to contact and/or deposit bone collected into the L5-S1 disc space area.

### CROSS-REFERENCES TO RELATED APPLICATIONS

This application claims the benefit of provisional application Ser. No. 62/483,974, filed on Apr. 11, 2017, incorporated herein by reference.

### FIELD OF THE INVENTION

The present invention relates to surgical implants, and more specifically, surgical implants utilized in association with spine surgery.

### BACKGROUND OF THE INVENTION

The present inventors have recognized the problem of creating a bone bridge between the L5 and S1 vertebral bodies during pre-sacral interbody fusion. Other solutions for the problem of creating fusion between the L5 and S1 vertebral bodies during pre-sacral interbody fusion exist in the prior art. These solutions, however, have failed to meet one or more unsolved needs recognized by the inventor because of still-remaining challenges. Moreover, previous implants intended to accomplish pre-sacral interbody fusion do not allow bone to grow into and through the implant. In addition, the previous implants do not allow bone or other materials such as autograft, allograft, bone cement, or some other type of bone biologic to effectively be deposited in the intervertebral disc space and or vertebral bodies. Other prior art pre-sacral interbody fusion implants likewise do not collect bone from the sacrum as they traverse through the sacrum for re-deposit into the L5-S1 interbody space.

### BRIEF DESCRIPTION OF FIGURES

FIG. 1 depicts a profile view of an embodiment of the invention.

FIG. 2 depicts an oblique top down view of an embodiment of the invention.

FIG. 3 depicts a cross-sectional view of an embodiment of the invention.

FIG. 4A and 4B each depict a profile view of alternative embodiments of the invention featuring alternative configurations of fenestrations located between the threads located on the exterior of the main body.

FIG. 4C depicts an embodiment of the invention featuring custom threading.

FIG. 5 depicts a side view of an embodiment of the invention featuring a single fenestration within the main body.

FIG. 6 depicts a perspective view of the fenestration within the main body in an embodiment of the invention.

FIG. 7 depicts an embodiment of the invention featuring small circular fenestrations located within the main body.

FIG. 8A depicts alternative embodiments of thread patterns comprising to be utilized in association with custom threading in various embodiments of the invention.

FIG. 8B depicts the “thread within a thread” type of custom threading utilized in an embodiment of the invention.

FIG. 9A depicts a side view featuring a plurality of fenestrations configured in a pattern parallel to the thread pattern positioned within the threads of the threading in an embodiment of the invention.

FIG. 9B depicts a plurality of fenestrations configured in a slotted scoop pattern in an embodiment of the invention such that the hole cut into the main body has an axis orthogonal to a plane that is tangent to the outer diameter of the threaded rod and rotated 0-45 degrees relative to another plane that is tangent to the outer diameter of the threaded rod and parallel to a plane that intersects the primary axis. In the embodiment depicted by FIG. 9B, the hole is positioned in the root of the threads around the rod on either opposite side of the threaded rod.

FIG. 9C depicts a plurality of fenestrations configured in a circular scoop pattern in an embodiment of the invention, wherein the fenestrations are staggered in order to maximize the cross-sectional area of the threaded rod in a plane that is orthogonal to the primary axis of the threaded rod in order to maximize the bending strength of the rod.

FIG. 10 depicts the end view incorporating a circular scoop in order to demonstrate the fenestration angle relative to the main body in an embodiment of the invention.

FIG. 11a depicts an embodiment of the main invention incorporating a slotted helix design. FIG. 11b depicts an embodiment of the main body of the invention featuring a slotted helix profile.

### BRIEF DESCRIPTION OF NUMERICAL REFERENCES IN FIGS.

1. Main Body in an embodiment of the invention.

2. Fenestration (optionally, for retaining bone graft) in an embodiment of the invention.

3. Cutting Edge For Harvesting Graft in an embodiment of the invention.

4. Tapered Distal Section in an embodiment of the invention.

5. Hex Driver Connection in an embodiment of the invention.

6. Custom Threading For Bone Engagement in an embodiment of the invention.

7. Chip Breaker in an embodiment of the invention.

8. Slotted Threading in an embodiment of the invention.

### DESCRIPTION OF THE INVENTION

The preferred embodiment of the present invention is described as a fenestrated non-distracting implant. In the preferred mode of use, the implant, in the preferred embodiment more precisely described as a fenestrated non-distracting implant, is utilized in association with fusion of the L5 and S1 vertebral bodies. Generally, the inventors intend for the implant to function via advancing through the sacral vertebral body by a method commonly described as “the pre-sacral approach” as known in the prior art, and as more specifically described in U.S. Pat. No. 6,575,979 which is hereby incorporated by reference in its entirety, through the L5-S1 interbody space, and into the L5 vertebral body. In the preferred method of use, during passage through the S1 vertebral body, fenestrations within the implant are configured to interact with the sacrum as the implant advances and collect bone tissue from the sacrum into the fenestrations. The implant, in the preferred embodiment may also be configured such as to deposit bone from the sacrum into the L5-S1 interbody space. The bone collected within the fenestrations of the implant may also come into contact with bone graft previously and/or subsequently placed within the L5-S1 interbody space. In alternative embodiments of the invention, the implant may be placed in other locations both within and outside of the body. The present inventors have contemplated that attributes of the invention allow for increased push-through and pull-out resistance and/or axial resistance in a variety of contexts both within and outside of the human body. In variants of embodiments of the invention, the implant may be adapted for placement across the sacro-iliac joint in association with sacro-iliac fusion. In other embodiments, the implant is placed through one or more vertebral bodies at locations other than the L5-S1 interbody junction, especially including, for instance, the L4-L5 interbody junction. In embodiments, aspects of an embodiment the invention may form a portion of a pedicle screw as known by one skilled in the art, more particularly on the screw portion of the pedicle screw, and/or wherein an embodiment of the invention as described herein replaces the threaded shank portion of a pedicle screw. In varying embodiments, the invention may incorporate any subset of, or all of, the following components: (1.) a main body, (2.) a fenestration for retaining bone graft, (3.) a cutting edge for harvesting graft, (4.) a tapered distal section, (5.) a hex driver connection, (6.) custom threading for bone engagement, (7.) a chip breaker, (8.) slotted threading.

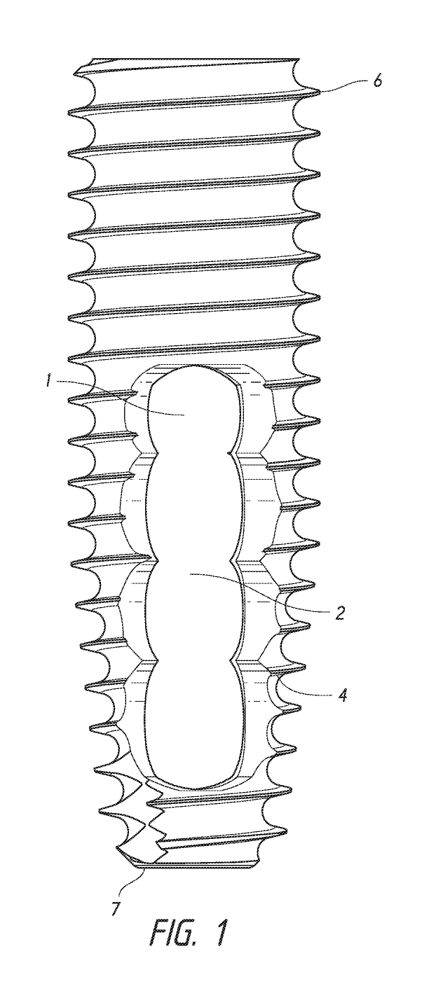

An embodiment of the invention incorporates a main body 1, as depicted by FIG. 1. The preferred embodiment of the main body 1 comprises the following approximate dimensions: 14 millimeters outer diameter at the widest point, 50 millimeters in length. Another embodiment of the main body 1 comprises the dimensions of 15.5 millimeters outer diameter at the widest point. The preferred embodiment of the main body 1 incorporates a medical grade titanium (Ti-6A1-4V ELI) in its composition. Embodiments may comprise commercially pure titanium, titanium configured through use of additive manufacturing and/or 3D printing, or polyetheretherkeytone (PEEK). Manufacturing methods used to create embodiments of the main body 1 may include traditional milling and lathing and 3D printed titanium, as known by one skilled in the art. The main body 1 in an embodiment of the invention is generally described as a substantially cylindrical construct surrounded by bone-engaging threading.

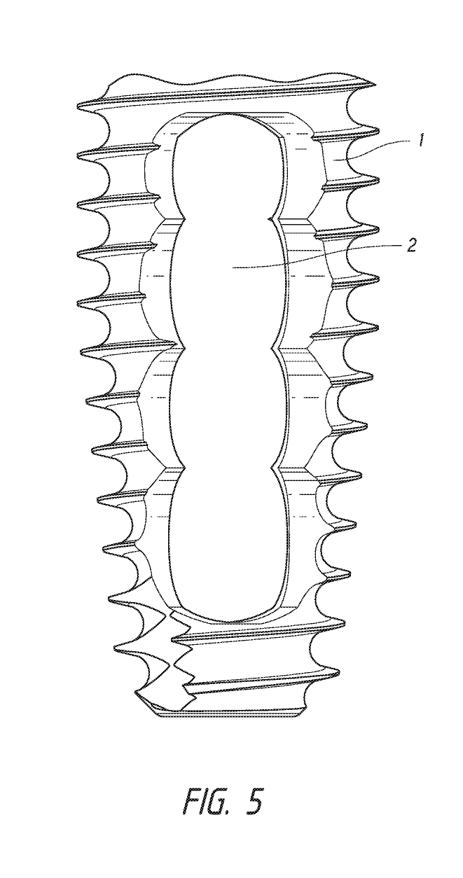

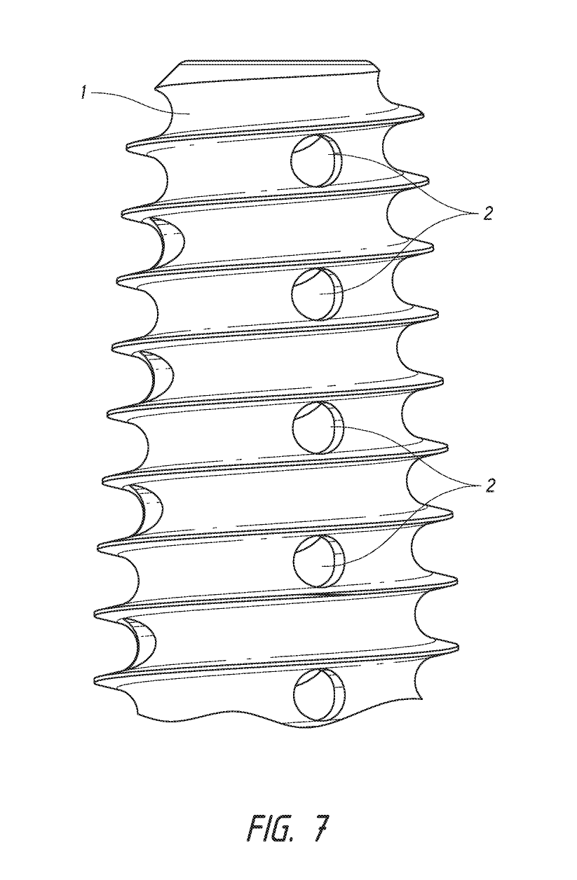

An embodiment of the invention incorporates one or more fenestrations for retaining bone graft 2. In an embodiment, such one or more fenestrations comprise a sub-component of a main body 1. Embodiments of said one or more fenestrations are depicted in at least FIGS. 1, 2, 3, 5 and 6, 7, 9a-c, 10 and 11a-b. In varying embodiments of the invention, one or more fenestrations are incorporated into the main body 1 such that they scoop or collect bone as the threaded rod is inserted into the prepared hole in the sacrum. In an embodiment, the bone collection occurs by rotating the threaded rod in a clockwise (right hand thread) fashion.

The preferred embodiment of a fenestration for retaining bone graft comprises the following dimensions: approximately 10 millimeters in width at the widest point and approximately 25 millimeters in height running along the height of the main body. The fenestration for retaining bone graft 2 in an embodiment of the invention is described as an aperture within the main body of the implant designed to collect and house bony material as the main body passes through and into the bony structures associated with the spinal column. The present inventors have recognized the advantages associated with avoiding reduction in thread surface area for resisting loads in the direction parallel to the primary axis of the threaded rod. Various embodiments of the invention allow for such advantages.

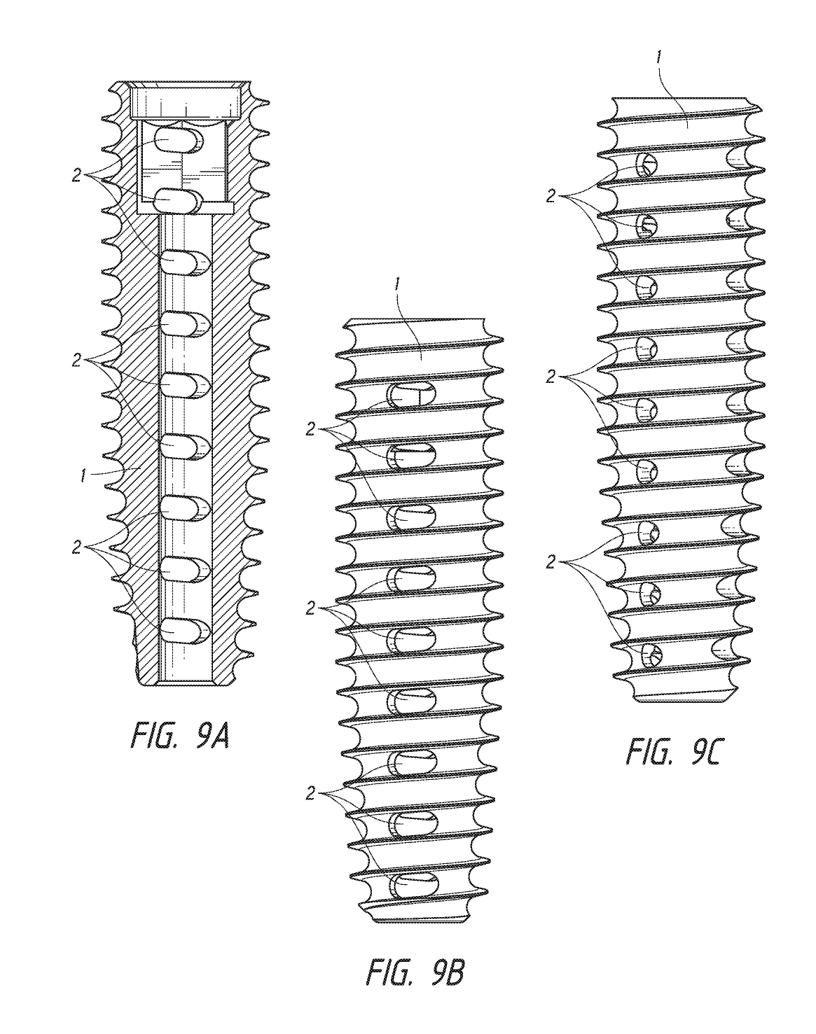

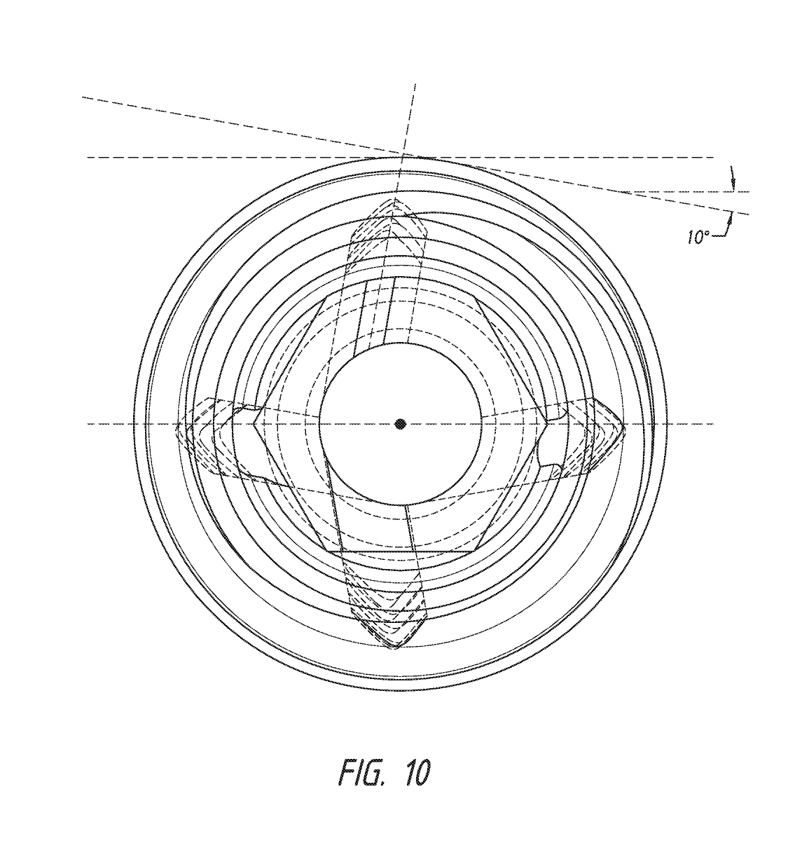

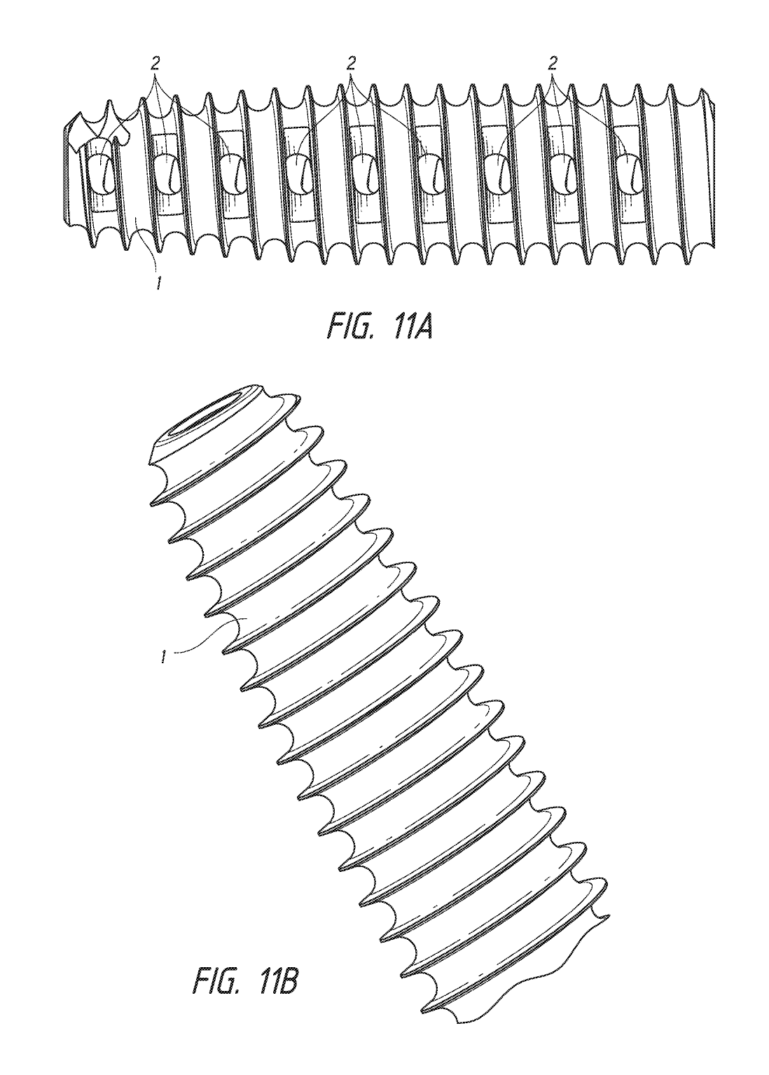

In an alternative embodiment featuring one or more fenestrations, as depicted within FIGS. 11a and 11b, the single fenestration or more than one fenestrations are cut such that the axis of the slot follows the helical pathway in the root of the thread. In an embodiment featuring such one or more fenestrations as described in the preceding sentence, the axis of the cut intersects the primary axis of the threaded rod and follows the helical pathway in the root of the thread for 0-0.5 revolutions around the threaded rod. In another alternative embodiment of the invention, the fenestration comprises a hole as depicted in FIG. 9c. In another alternative embodiment of the invention, such fenestration comprises a slot as depicted in FIG. 9b. In embodiments of the invention such as those described in the preceding two sentences, the one or more fenestrations are oriented such that their axis is orthogonal to a plane that is tangent to the outer diameter of the threaded rod and rotated 0-45 degrees relative to the Paratan Plane tangent to the outer diameter of the implant (referred to as the “Paratan Plane” and further described in FIG. 10). In an embodiment, the one or more fenestrations are positioned in the root of the threads around the rod on either opposite sides of the threaded rod in a “slotted scoop” concept depicted in FIG. 9b. In an alternative embodiment, the one or more fenestrations are positioned in every quadrant such that the fenestrations are staggered in order to maximize the cross sectional area of the threaded rod in a plane that is orthogonal to the primary axis of the threaded rod in a “circular scoop” depicted in FIG. 9c and FIG. 10. The present inventors have recognized that such design specifically maximizes the bending strength of the rod.

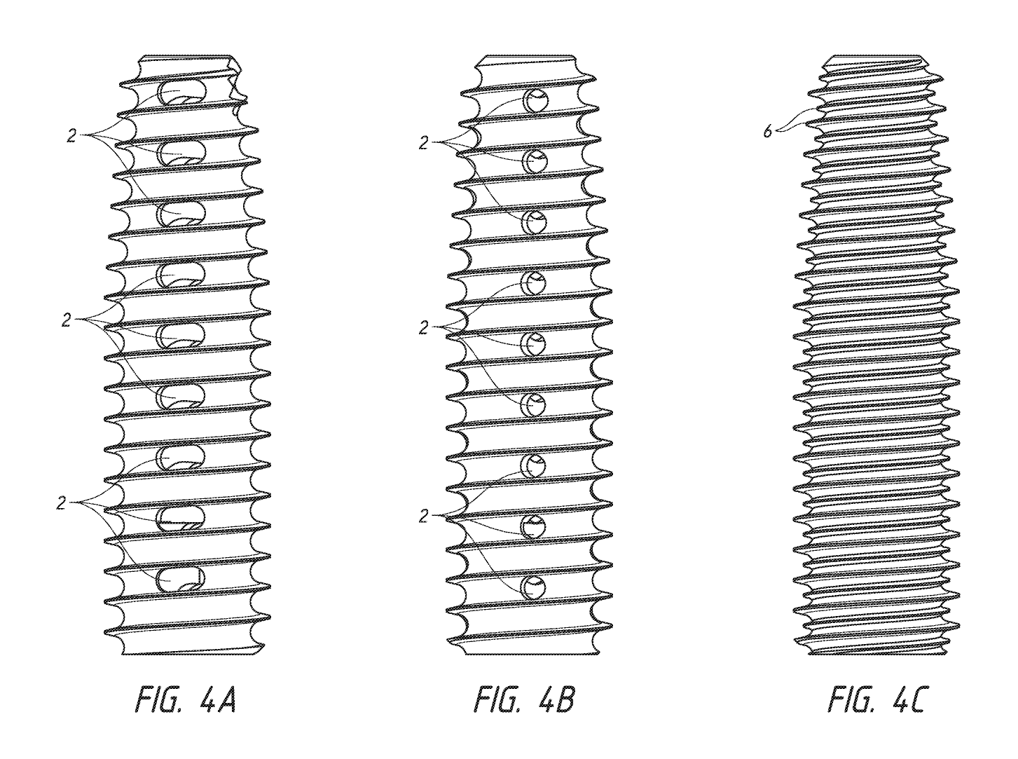

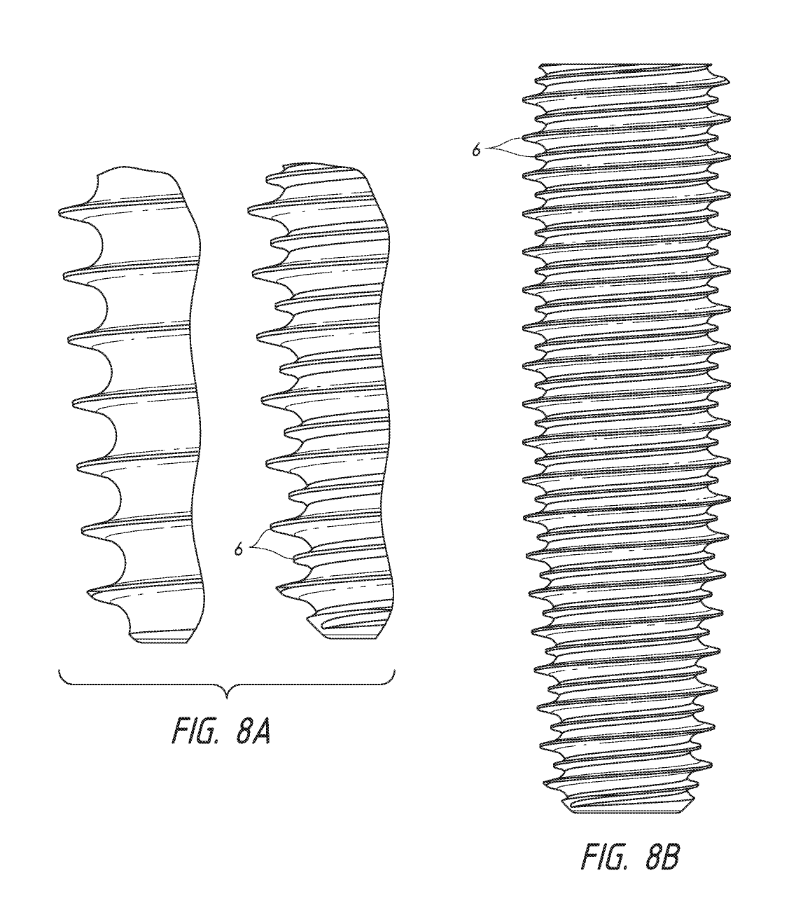

In an embodiment of the invention, the entire construct comprises a main body 1, a major thread, and a minor thread located within the root of the major thread, optionally with or without fenestrations in the main body, as depicted in FIGS. 4C and 8B. The present inventor has recognized application for such embodiment in contexts within spine surgery, broader surgery, and for use outside of the body. In such embodiment, the entire construct could appropriately be described as a “screw” as understood by one skilled in the art. An embodiment incorporates threading comprising a minor thread and a major thread where said minor thread has a minor diameter that is shared with the minor diameter of the major thread. In an embodiment, said minor thread has a major diameter that is greater than the minor diameter of the major thread and less than the major diameter of the major thread. In an alternative embodiment where the minor thread follows a different diametric helical pathway than the major thread. In an embodiment, the minor thread and major thread have the same thread pitch. In an embodiment, the minor thread only exists along one or more selected portions of the major thread.

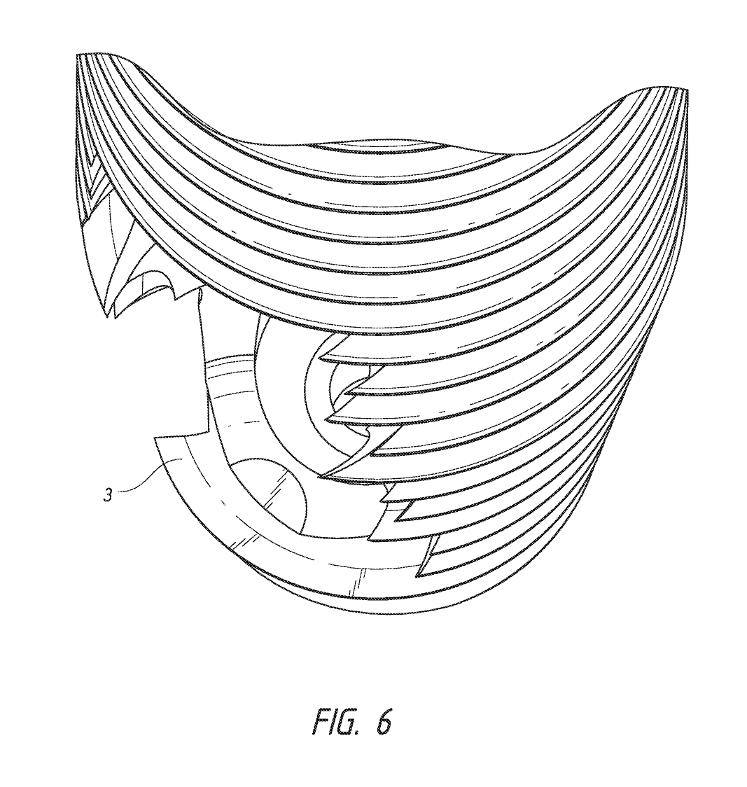

An embodiment of the invention incorporates a cutting edge for harvesting graft 3, a sub-component of a main body 1, as depicted by FIG. 6. A cutting edge for harvesting graft 3 in an embodiment of the invention is described as an angled cut in the main body at the leading edge of the fenestration designed to shave bone material into the fenestration of the main body from one or more bony structures of the spine during insertion through one or more bony structures. In an embodiment, the cutting edge for harvesting graft 3 is described as a sub-component of a main body 1.

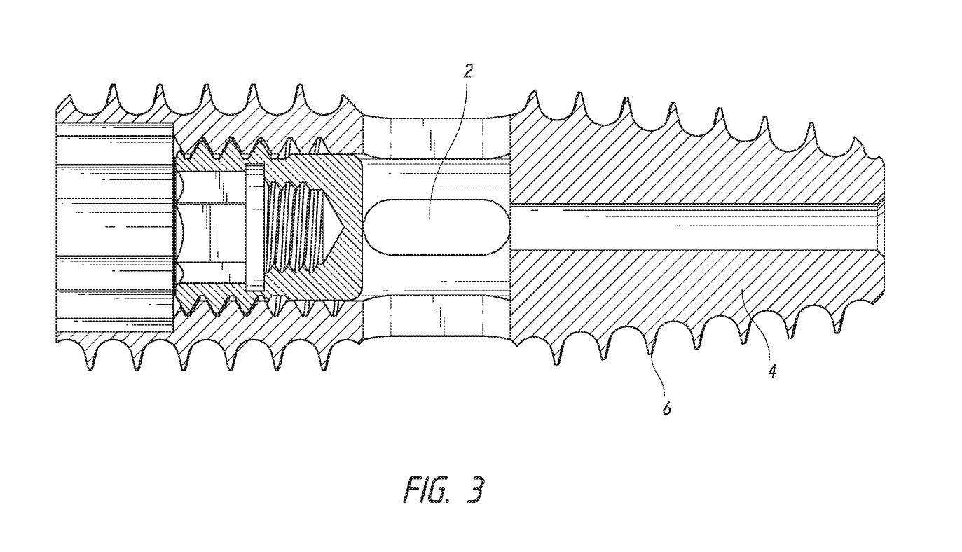

An embodiment of the invention incorporates a custom threading for bone engagement 6, a sub-component of a main body 1, as depicted by FIGS. 3 and 8. The preferred embodiment of the custom threading for bone engagement 6 comprises the following dimensions: a larger diameter (approximately 2 millimeters) thread and a smaller diameter thread (approximately I millimeter) within the root of the larger thread along the same pitch line. The preferred embodiment of the custom threading for bone engagement 6 incorporates a medical grade titanium (Ti-6A1-4 V ELI) as the primary material in its composition. A custom threading for bone engagement 6 in an embodiment of the invention is described as a thread within a thread. Embodiments featuring the thread within a thread design are depicted in FIGS. 8a and 8b. The present inventors have recognized that the thread within a thread design in varying embodiments of the invention increases surface area and mechanical engagement. This design may allow for tap-like flutes to be cut into the threads without reducing the overall surface area contacting bone which provides increased resistance to axial forces. An embodiment of the custom threading for bone engagement 6 may alternatively incorporate threading that has angled holes within the threading to allow for the harvesting of bone graft through holes within the main body between the threading.

The present inventors have recognized that in varying embodiments of the invention featuring a thread within a thread design, such as those depicted by FIGS. 8a and 8b, the increased thread surface area permits the implant to better resist loads in a direction parallel to the primary axis of the threaded rod. In such embodiments, the threaded rod consists of a major thread and a minor thread such that the major thread has a large enough thread profile to contain a smaller minor thread. In such embodiments, the major and minor thread share the same minor diameter. In such embodiments, the major thread has a major diameter that is larger than that of the major diameter of the minor thread. In such embodiments, the thread pitch of the major and minor thread are the same. The present inventors have recognized that design contemplated in such embodiments could be incorporated into a dual lead thread design. The present inventors have recognized that in such embodiments the minor thread could be applied along the length of all or part of the thread rod in order to increase surface area or “thread purchase” in specific desired areas. The preferred method of manufacture of such embodiments is additive manufacturing also referred to 3D printing, as recognizable by one skilled in the art, as the present inventors have recognized that conventional machining methods could increase the difficulty of the fabrication of the thread within a thread feature.

An embodiment of the invention incorporates a tapered distal section 4, as depicted by FIGS. 1 and 3. The preferred embodiment of the tapered distal section 4 comprises the following dimensions: approximately 15-20 millimeters in length with a final major diameter of 11 millimeters, with a 4 degree angle of tapering. The preferred embodiment of the tapered distal section 4, incorporates a medical grade titanium (Ti-6A1-4V ELI) in its composition. A tapered distal section 4 in an embodiment of the invention is described as a portion of the main body designed to engage bone and seat in the L5 vertebra. In an embodiment of the invention, the tapered distal section 4 and a main body 1 are related to one another in such embodiment as the tapered distal section is a portion of the main body, designed to ultimately anchor into the L5 vertebral body. An embodiment of the invention incorporates a chip breaker 7, a sub-component of a tapered distal section 4. A chip breaker 7 in an embodiment of the invention allows for a cutting flute to self-tap the bone during insertion, a process as one skilled in the art would recognize, instead of requiring the use of a tapping drill during insertion. In an embodiment of the invention, a chip breaker 7 and a tapered distal section 4 are proximal. In an embodiment, the chip breaker 7 is a sub-component of a tapered distal section 4.

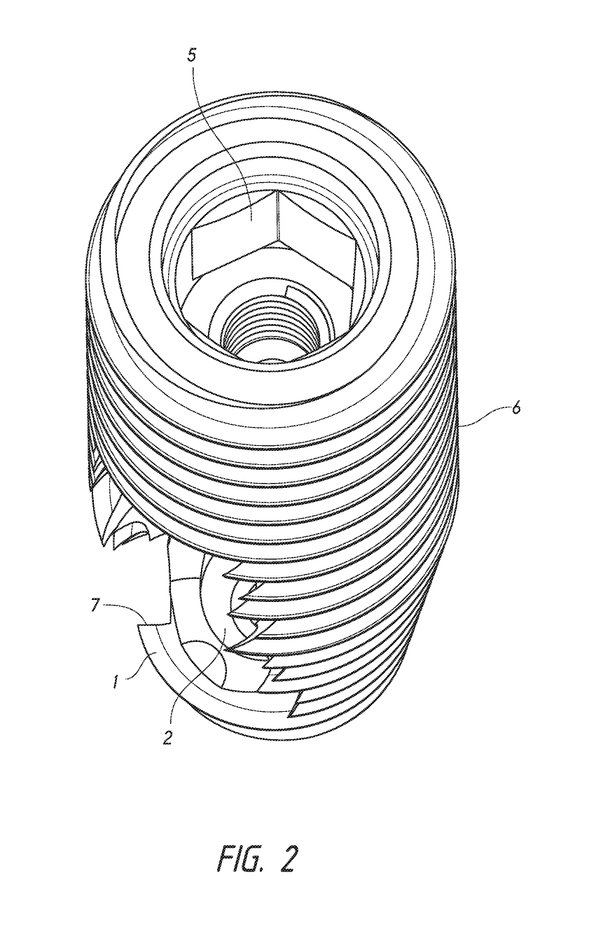

An embodiment of the invention incorporates a hex driver connection 5, as depicted in FIG. 2. The preferred embodiment of the hex driver connection 5 comprises the following dimensions: approximately 9 millimeter circular extrusion giving way to an approximately 7 millimeter internal hex connection. A hex driver connection 5 in an embodiment of the invention is described as a female connection point with a hexagonally-shaped cavity to accommodate a male driver with a hexagonally-shaped protrusion. The hex driver connection is designed to facilitate transmission of torque from the driver to the implant. In an embodiment of the invention, a hex driver connection 5 and a main body 1 are related to one another in such embodiment as the main body accommodates the hex driver connection 5. The hex driver connection 5 forms part of the main body 1 proximal to the surgeon to accommodate the instrumentation utilized to place the implant into the body.

An embodiment of the invention incorporates a slotted threading 8, as depicted by FIG. 4. In an embodiment of the invention, a slotted threading 8 and a custom threading for bone engagement 6 are related. A slotted threading 8 and a custom threading for bone engagement 6 are related to one another in such embodiment as the slotted threading exists in such embodiment within the threads generally parallel to and along substantially the same path of the threading.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has,” “having,” “includes,” “including,” “contains,” “containing,” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. The terms “coupled” and “linked” as used herein is defined as connected, although not necessarily directly and not necessarily mechanically. A device or structure that is “configured” in a certain way is configured in at least that way, but may also be configured in ways that are not listed. Also, the sequence of steps in a flow diagram or elements in the claims, even when preceded by a letter does not imply or require that sequence.

1. An implant for use in spinal surgery, comprising:
a main body;
at least one fenestration; and
a cutting edge.

2. The implant of claim 1, further comprising threading.

3. The implant of claim 2, with at least one fenestration positioned between the threads of said threading.

4. The implant of claim 2, where the axis of the fenestration does not intersect the primary axis of the main body

5. The implant of claim 2, where the axis of the fenestration is orthogonal to a plane that is tangent to the diameter of the main body.

6. The implant of claim 2, where the axis of the fenestration is orthogonal to a plane that is tangent to the diameter of the main body AND is positioned at an angle to a plane that crosses through the primary axis of the main body.

7. The implant of claim 1, manufactured via additive manufacturing processes.

8. The implant of claim 1, further comprising a tapered distal section.

9. The implant of claim 4, said tapered distal section comprising a chip breaker.

10. The implant of claim 1, further comprising a hex driver connection.

11. The implant of claim 1, configured for placement via the pre-sacral approach.

12. The implant of claim 1, configured for placement across the sacro-iliac joint.

13. A screw, comprising:
a main body;
a major thread;
a minor thread located within the root of the major thread.

14. The screw of claim 13, where said minor thread has a minor diameter that is shared with the minor diameter of the major thread

15. The screw of claim 13, where said minor thread has a major diameter that is greater than the minor diameter of the major thread and less than the major diameter of the major thread.

16. The screw of claim 13, where the minor thread follows a different diametric helical pathway than the major thread.

17. The screw of claim 13, where the minor thread and major thread have the same thread pitch.

18. The screw of claim 12, where the minor thread only exists along one or more selected portions of the major thread.
